(19) United States
(12) Patent Application Publication
(10) Pub. No.: US 2009/0062747 A1
(43) Pub. Date: Mar. 5, 2009

(54) Systems and methods for metered delivery of medication using stored energy

(75) Inventor: Tom A. Saul, El Granada, CA (US)

Correspondence Address: GRAYBEAL JACKSON HALEY LLP, Suite. 350, 155-108th Avenue N.E., Bellevue, WA, 98004-5973, US

(73) Assignee: Seattle Medical Technologies

(21) Appl. No.: 11/897,534

(22) Filed: Aug. 29, 2007

(51) Int. Cl. A61M 5/00

(52) U.S. Cl. 604/207

(57) ABSTRACT

Systems and methods for storing and dispensing a fluid on-demand includes a reservoir; an energy storage device coupled to the reservoir to store energy, wherein the energy is mechanically generated by a user's action; a cannula assembly adapted to be positioned on a patient and coupled to the reservoir prior to use; and a sequencer coupled to the reservoir and the energy storage device to allow the fluid to be dispensed upon command.

### BACKGROUND

The present invention relates to a portable drug delivery system.

Diabetes mellitus, more commonly known as diabetes, is a disease in which the body does not produce and/or properly use insulin, a hormone that aids the body in converting sugars and other foods into energy. Several types of diabetes exist. Insulin dependent diabetes mellitus (IDDM), commonly referred to as Type 1 diabetes, results from an auto-immune disease that affects the islets of Langerhans, destroying the body's ability to produce insulin. Type 1 diabetes may affect as many as 1 million people in the United States. Non-insulin dependent diabetes mellitus (NIDDM), commonly referred to as Type 2 diabetes, is a metabolic disorder resulting from the body's inability to produce enough insulin or properly use the insulin produced. Roughly 90 percent of all diabetic individuals in the United States suffer from Type 2 diabetes, which is usually associated with obesity and a sedentary lifestyle.

Diabetes is typically treated by monitoring the glucose level in the body through blood and/or urine sampling and attempting to control the level of glucose in the body using a combination of diet and parenteral injections of insulin. Parenteral injections, such as subcutaneous and intramuscular injections, deliver insulin to the peripheral system.

Insulin delivery has been dominated by subcutaneous injections of both long acting insulin to cover the basal needs of the patient and by short acting insulin to compensate for meals and snacks. Recently, the development of electronic, external insulin infusion pumps has allowed the continuous infusion of fast acting insulin for the maintenance of the basal needs as well as the compensatory doses for meals and snacks. These infusion systems have shown to improve control of blood glucose levels, however, they suffer the drawbacks of size, cost, and complexity, which prevents many patients from accepting this technology over the standard subcutaneous injections. These pumps are electronically controlled and must be programmed to supply the desired amounts of basal and bolus insulin.

### SUMMARY

In one aspect, an apparatus to store and dispense a fluid on-demand includes a reservoir; an energy storage device coupled to the reservoir to store energy for dispensing the fluid, wherein the energy is mechanically generated by a user's action; a cannula assembly adapted to be positioned on a patient and coupled to the reservoir prior to use; and a sequencer coupled to the reservoir and the energy storage device to dispense the fluid upon command.

Implementations of the above aspect may include one or more of the following. A metering assembly can be connected to the reservoir. The metering assembly can include an input valve; a metering chamber coupled to the input valve; and an output valve coupled to the metering chamber. The reservoir can be configured as a ring. The reservoir can be a rolling bellows. The metering assembly can be centrally located or encircled by the reservoir. The energy storage device can store energy when the reservoir is filled or when the reservoir is placed on the patient. The energy storage device can be a spring wound or compressed by the user's action. An interlocked user interface can be actuated by a user to dispense fluid or medication. The interlocked user interface can include an energy delivery button and an energy release button. The energy delivery button can charge an energy storage device such as by winding or compressing a spring. The release button allows energy to be discharged from an dispensing energy storage device to dispense the fluid. The spring can provide energy to activate the metering chamber. A metering spring can provide energy to activate the metering chamber. A dispensing feedback unit can indicate fluid dispensing. The cannula assembly can be one of: a cannula, a micro-needle, a needle. A receiving port can be provided on the device to receive a second medication that is different from medication stored in the reservoir. A user viewable gauge can be provided on the reservoir to show remaining medication. One or more energy storage devices can be used singly or together: one energy storage device can provide energy to dispense the fluid or medication through the cannula assembly, and another energy storage device can be used to move valves in a predetermined sequence.

In another aspect, systems and methods for delivering medication to a patient are disclosed. The system has three assemblies which are mated during use: a reservoir, a sequencer/monitor, and an insertion set.

In yet another aspect, a method for dispensing medication includes storing medication in a reservoir; and storing energy in a storage device coupled to the reservoir and using the energy to transfer an amount of medication through an input valve to fill a metering reservoir having a predetermined volume, to transfer the predetermined volume of medication through an output valve and to transfer the predetermined volume of medication to a patient.

In a further aspect, a method for dispensing a medication includes receiving the medication across an input septum and storing the medication in a reservoir; storing energy in a storage device coupled to the reservoir and using the energy to, upon user actuation, transfer medication through an input valve into a metering reservoir having a predetermined volume; and delivering the medication to the patient through a cannula.

Implementations of the above method may include one or more of the following. The predetermined volume is user-adjustable. The predetermined volume can be transferred through a cannula assembly. Medication can be delivered across an input septum and storing the medication in a reservoir. The reservoir can be filled with fluid or medication using a syringe. The stored mechanical energy can be used to dispense medication. The system can store sufficient mechanical energy to deliver one or more doses or to provide a basil delivery. The system can receive a second medication at a receiving port. A user viewable gauge can be used to show a user the remaining medication. The predetermined amount of medication can delivered to the patient through a cannula, a micro-needle, or a needle. The system can count user actuations, determine a total dispensed dosage, and display the total dispensed dosage.

In yet another aspect, a method for dispensing a medication includes receiving the medication across an input septum and storing the medication in a pressurized reservoir containing energy to transfer the medication; upon user actuation, transferring medication through an input valve into a metering reservoir having a predetermined volume; and delivering the medication to the patient through a cannula using the energy from the pressurized reservoir.

Implementation of the above aspect may include one or more of the following. The predetermined volume of medication can be moved through an output valve.

In another aspect, an apparatus for dispensing fluid includes a pressurized reservoir; a metering chamber; an output interface in fluid communication with the metering chamber; a cannula assembly coupled to the output interface, and a sequencer coupled to the pressurized reservoir to dispense the fluid.

In another aspect, an apparatus for dispensing a fluid includes a pressurized reservoir; a metering assembly including: an input valve coupled to the reservoir; a metering chamber coupled to the input valve; and an output valve coupled to the metering chamber; and a cannula assembly in fluid communication with the output valve.

In a further aspect, a medication dispenser includes an input interface across which a medication charge is received; a pressurized reservoir in fluid communication with the input interface to store medication therein; and a metering chamber in fluid communication with the reservoir to define and facilitate dispensing a predetermined amount of mediation to a patient.

Implementations of the above aspect may include one or more of the following. A reservoir drive can drive the reservoir. The reservoir drive can be pressurized by one of: a spring, a gas source, or a phase change material. The reservoir drive can store energy from each user actuation. The reservoir drive can store energy when the dispenser is attached to a sequencer. The reservoir drive can be charged prior to when the dispenser is attached to a sequencer. The metering chamber can be charged by an external energy source including one of: a spring, a gas source, a phase change material. In another embodiment, the metering chamber has first and second chambers, wherein alternately a flow of medication into one chamber drives medication in the other chamber into a patient. One or more control valves coupled to the metering chamber to sequence medication flow. A cannula assembly can be connected to the reservoir output. The cannula assembly comprises one of: a cannula, a micro-needle, a needle and can be positioned on a patient using an applicator. A fill indicator can be connected to the reservoir. A clear window can be provided to allow users to view a medication level in the reservoir. A dosing indicator can be connected to the reservoir to indicate a dispensed medication dosage. An interlocked user interface can be used to request fluid dispensing. The interlocked user interface can have two buttons and the patient can activate the two buttons in a predetermined sequence to dispense medication. A clock or a timer can work with the interlocked user interface to measure dispensed dosages over a particular time interval. The reservoir can have a medication storage volume having a first configuration where the medication storage volume is maximized and a second configuration where the medication storage volume is minimized. The reservoir can be a rolling bellows, among others. In one embodiment, the system has an energy storage device that drives a medication storage device, herein termed reservoir, to maintain pressure on the medication in the medication storage device. The system also provides a metering chamber that receives medication from the reservoir. The metering device can be connected to a different energy storage device which stores energy provided by the reservoir as the metering chamber is filled and which provides a driving force to empty the metering chamber. A set of valves can be used to control fluid input and output from the metering chamber. A sequencer can activate the metering device; and interface hardware can be used to connect the output from the metering chamber to an appropriate delivery location in the patient and allow the delivery of the precisely metered bolus of medication contained in the metering chamber to the patient.

In another embodiment, the system can be configured as one to four or more assemblies which are mated in use. The assemblies can include a disposable unit incorporating a reservoir, a metering chamber, and control valves; a sequencer/monitor incorporating a user interface, driving means for control valves; and interface hardware such as an output needle, and an insertion set.

The metering chamber can be externally driven or can be internally driven. In one embodiment, a spring can be charged by an influx of pressurized insulin from the reservoir. Alternatively, the metering chamber may provide two chambers separated by a flexible membrane where the influx of insulin in one chamber drives a previous charge of medication out of the other chamber to the patient. The sequencer may incorporate a safety interlocked user interface which minimizes the risk of inadvertently activating the delivery of an unneeded bolus of medication. For example, a two-button user interface can be used with one button for release and one button for activation. The user interface can also capture energy provided by the user to charge the energy storage device. The interlocked user interface control can be used to drive the valves and can be mechanical, electrical, or a combination thereof. A mechanical or electrical clock can also be provided to provide regularly scheduled boluses thereby providing for basil delivery. A data storage device or other suitable memory function can be incorporated in the sequencer to provide time stamped information and/or medication dosages administered over a predetermined period. An adhesive backing can be provided to attach the system to the patient. Seals can be used in one embodiment for assuring sterility. Further, the sequencer/monitor as well as the disposable device can be vacuum sealed in their empty configuration.

Advantages of the system may include one or more of the following. The system provides a minimally-perceptible insulin pump that is manually activated by the user and that is small enough to hide under the user's clothing. The system is inexpensive and convenient to use. The system also provides a convenient, secure and inconspicuous user interface to dispense medication as needed. The system can be inconspicuously placed under the patient's clothing and is always available for dispensing medication. The system can be inconspicuously activated without hinting to others that the patient is in the process of injecting medication. The system also provides the ability to provide a basil delivery of medication such as insulin. The energy required to deliver the fluid or medication is stored as mechanical energy and transferred to the reservoir, while the energy necessary to sequence the action of delivery can be provided by the same energy source or a secondary source and can be either mechanical or electrical energy. Overall, the system improves the level of care for patients such as diabetic patients by providing on-demand medication to minimize episodes of over or under treatment.

### BRIEF DESCRIPTION OF THE DRAWINGS

The invention, together with further features and advantages thereof, may best be understood by making reference to the following description taken in conjunction with the accompanying drawings, in the several figures of which like reference numerals identify identical elements, and wherein:

FIG. 1 shows a block diagram depiction of a first embodiment of a metering system to deliver medication.

FIG. 2 shows a block diagram depiction of a second embodiment of a metering system to deliver medication.

FIG. 3 shows a top perspective view of an exemplary disposable portion of a medication delivery device.

FIG. 4 shows a bottom perspective view of part of the disposable portion of a medication delivery device of FIG. 3.

FIGS. 5 and 6 show bottom and top perspective cross-sectional views, respectively, of the device of FIG. 3.

FIG. 7 shows another cross-sectional view of the device as depicted in FIG. 4.

FIGS. 8-10 show an exemplary sequencer/monitor device in cooperation with the disposable device of FIG. 3.

FIG. 11 shows a processor that controls one or more solenoids in FIGS. 8-10.

FIGS. 12A-12D show an exemplary process for deploying the device of FIG. 3.

FIG. 13 shows a top perspective view of a disposable for a second exemplary disposable portion of a medication delivery device.

FIG. 14 shows a bottom view of the device of FIG. 13 with its base plate removed.

FIGS. 15 and 16 show exemplary cross-sectional views of the device of FIG. 13.

FIG. 17, Shows an exemplary cross-sectional view illustrating the metering chamber of the device of FIG. 13.

FIGS. 18, 19, and 20 show exemplary cross-sectional views illustrating the operation of an output valve of the device of FIG. 13.

FIGS. 21-25 show various views of a portion of an exemplary valve control system for the device of FIG. 13.

FIGS. 26-30 illustrate an exemplary embodiment that receives energy from the patient and that stores and releases mechanical energy to dispense medication to the patient.

FIG. 31 shows an enclosure or housing for the embodiment of FIGS. 26-30 with openings to accommodate an energy input member and a catch and release button.

FIG. 32 shows a cross-sectional view of the entire device in its enclosure or housing.

FIGS. 33-35 illustrate additional views of the exemplary embodiment of a device that receives energy from the patient and that stores and releases mechanical energy to dispense medication to the patient.

### DESCRIPTION

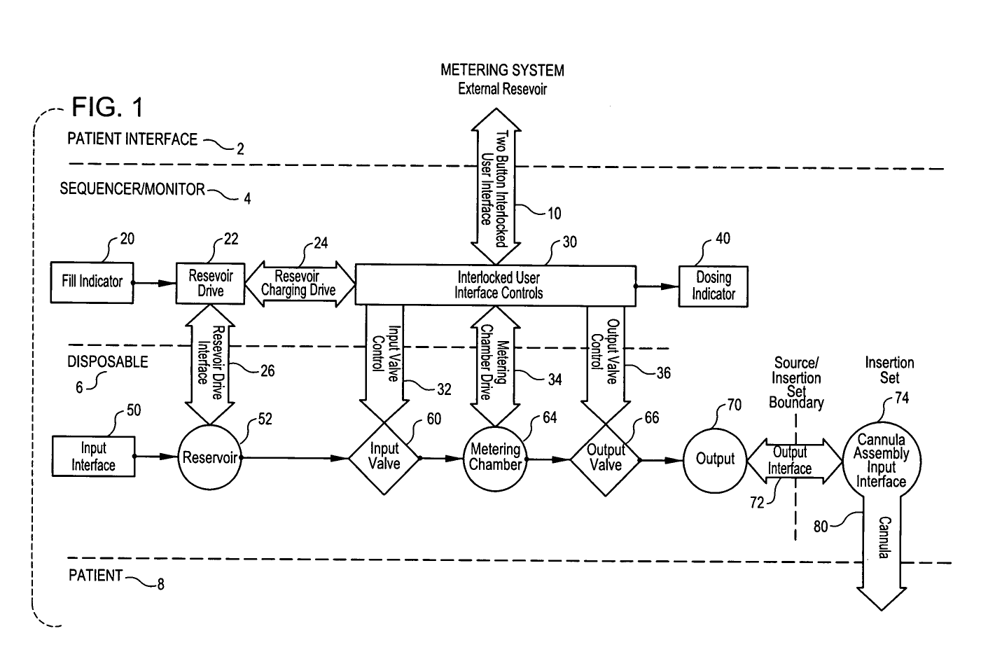

FIG. 1 shows a first embodiment of an exemplary metering system 1A for providing medication to a patient. The metering system 1A includes a patient interface portion 2 that receives input(s) from a patient and in turn communicates patient commands to a sequencer/monitor portion 4. The sequencer/monitor portion 4 communicates with a disposable portion 6 and activates the disposable portion 6 to release medication to the patient.

In one implementation, medication is released through the patient's skin 8 using a suitable medication dispensing device such as a needle, a micro needle, a cannula, or other suitable dispensing devices. By separating the metering system into the sequencer/monitor portion 4 and the disposable portion 6, the operating cost of the metering system 1A can be reduced since components that can be reused are housed in the sequencer/monitor portion 4, while the disposable portion 6 contains medication that, after usage and/or depletion, can be replaced with another compatible disposable portion 6.

In one embodiment, the patient interface 2 has an interlocked user interface such as a two button interlocked user interface 10. As described in more detail below, in one embodiment, the patient activates more than one button simultaneously or in series, to request the metering system to deliver medication such as insulin into the patient. The two button interlocked interface 10 provides enhanced safety to protect against accidental dosing by the patient. In other embodiments, the interlocked user interface can have one or more buttons that are actuated by the user in a predetermined sequence. In yet other embodiments, the interlocked user interface can be a single button such as a lever that is moved in a predetermined direction by the user or patient and then pushed in for actuation. The mechanical actuation energy provided by the user or patient can be stored and can then be used to power the delivery of the medication. The energy can be stored in an energy storage device for subsequent delivery, or alternatively can be immediately released when the user completes the predetermined actuation sequence.

Turning now to the sequencer/monitor portion 4, a fill indicator 20 provides a visual display of a remaining medication indication. In one embodiment, the fill indicator 20 is in fluid communication with a reservoir drive 22 and the level of remaining medication in the reservoir drive 22 can be estimated using a user viewable gauge formed on a reservoir made of a clear (see-through) material.

In the embodiment of FIG. 1, energy needed for emptying the metering chamber is provided by a second storage device that operates on energy previously stored in the reservoir drive. The reservoir drive 22 can receive energy from an optional reservoir charging drive 24, which is charged or activated by an interlocked user interface control system 30, or may be charged by the user when interfacing the disposable portion 6 with the sequencer. The control system 30 also provides a dosing indicator 40 that provides visual or audible indication to the patient on the number of dosages that the patient has injected over a predetermined period of time. In one embodiment, the system 30 provides an inconspicuous feedback to the patient that a particular dosage injection was successful. This feedback can be in the form of a tactile vibration, a audible sound, or a visual display.

The disposable portion 6 includes an input interface unit 50 which communicates with a reservoir 52. The reservoir 52 is powered by the reservoir drive 22 through a reservoir drive interface 26. The output of the reservoir 52 is directed through an input valve 60 to fill a metering chamber 64 with medication to its predetermined volume. Through an output valve 66, the metering chamber 64 is controllably allowed to drain through an output chamber 70. The control system 30 controls the input valve 60, optionally the metering chamber 64, and output valve 66 through an input valve control 32, an optional adjustable metering chamber drive 34 and an output valve control 36, respectively. In one embodiment that incorporates a timing mechanism in the interlocked user interface controls 30, the metering chamber 64 can operate independently of the two button interlocked user interface 10, and thereby provide for basal delivery.

The output chamber 70 is on one side of a source/cannula assembly boundary, and through an output interface 72, provides medication into a cannula assembly input interface 74 on the other side of the boundary. In one embodiment, the cannula assembly input interface 74 communicates with a cannula 80 to inject medication into the patient through the patient's skin 8.

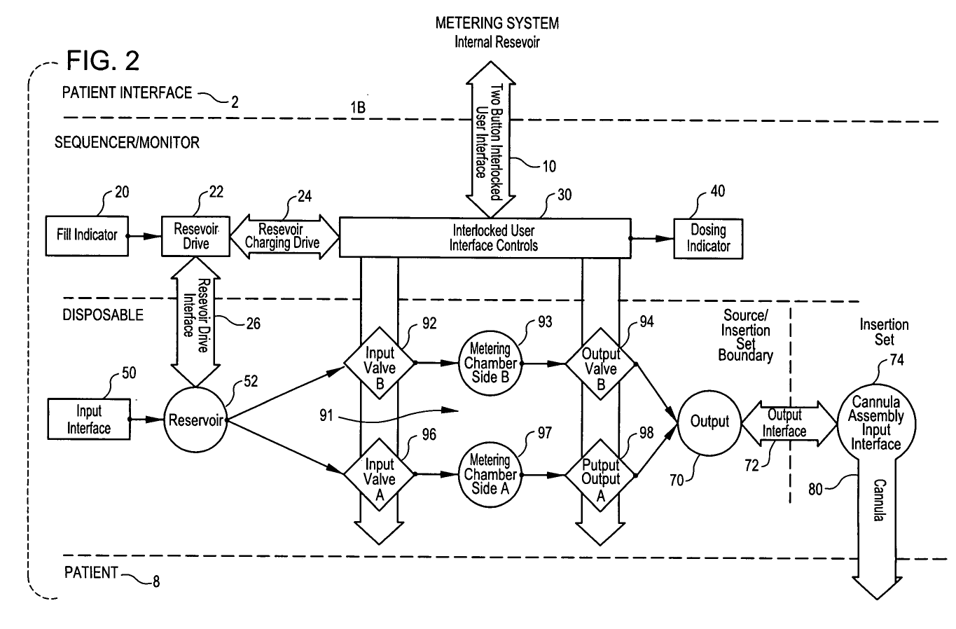

FIG. 2 shows a second embodiment of an exemplary metering system 1B for providing medication to a patient where energy from the reservoir is used to empty the metering chamber and to dispense medication. The metering system 1B is similar to the metering system 1A, but does not require an additional energy storage device in addition to the one coupled to the reservoir 52.

In the system of FIG. 2, a series of control valves are used for sequencing inflow and outflow from the metering chamber. The reservoir 52 communicates with first and second input valves 92 and 96. The first input valve 92 allows medication from the reservoir 52 to fill a first side 93 of a metering chamber 91 upon command from the interface control system 30.

The interface control 30 cycles through the following sequencing states. In the first state, all valves are closed to provide a safe starting and resting point. Next, valves 92 and 98 are opened to allow the chamber first side 93 to fill (thus draining the chamber 97). Next, the valves 92 and 98 are closed and valves 96 and 94 are opened thereby allowing the second side 97 of chamber 91 to fill, thus draining the chamber first side 93. Finally, all valves are closed to provide a safe resting point.

An alternative view of the valve sequencing may be seen as follows. Through the output valve 94, the metering chamber first side 93 drains medication through the output of the output chamber 70 upon command from the interface control portion 30. Similarly, the second input valve 96 allows medication from the reservoir 52 to fill the second side 97 of metering chamber 91 upon command from the interface control system 30. The metering chamber second side 97 in turn drains through the output of the output chamber 70 upon command from the interface control system 30 to the output valve 98.

The disposable portion 6 provides the input interface 50 for the patient, his or her physician, nurse, or a medical staff member, to fill the reservoir 52 with medication such as insulin, for example. In one embodiment the reservoir 52 can incorporate a rigid portion and flexible portion. In this embodiment, the reservoir 52 flexible portion may be bi-stable such that in one stable configuration the internal volume of the reservoir 52 is at its maximum and in the other stable confirmation the internal volume of the reservoir is at its minimum.

The reservoir drive 22 or charging component for the reservoir may store energy in various ways such as energy stored in a spring, energy stored in a material such as an artificial muscle, or energy stored in a pressure source such as a pressurized gas or a pressurized gas in conjunction with its liquid phase, among others. The reservoir drive 22 can be charged incrementally, during each activation by the patient, which can provide enough charge to complete one medication dispensing cycle. The reservoir drive 22 can also be charged when the disposable portion 6 and the sequencer/monitor portion 4 are snapped together or otherwise attached together by the patient. Alternatively, the energy storage device may be shipped in a pre charged condition, where upon interfacing to the disposable the energy is released to the reservoir. The charging source can be contained in the disposable portion 6 or in the reusable portion sequencer/monitor portion 4. When the charging source is incorporated in the disposable portion 6, the reservoir drive 22 may alternatively be charged by the action of incorporating medication in the reservoir, or be charged at the time of manufacture and released after filling. In one embodiment, the configuration can preclude reuse of the disposable portion 6 and assure sterility of the system 1A or 1B at the time of use.

The metering chamber 64 can be externally driven or can be internally driven. In one embodiment, a spring can be charged by the influx of pressurized insulin from the reservoir 52 (external drive configuration of FIG. 1). In the embodiment of FIG. 2, the influx of pressurized insulin, from the pressurized reservoir 52 into one chamber, drives a previous charge of medication out of the other chamber.

A safety interlocked user interface can be used. For example, a two-button user interface can be used with one configuration for release and one configuration for activation. Further, either configuration can capture user-generated energy to provide additional charges to the system. The interlocked user interface controls 30 can incorporate valve drives and can be mechanical, electrical, or a combination thereof. A mechanical or electrical clock can also be provided for basil delivery. The clock can provide time stamped information or a sum of doses administered in the last time period of defined duration. The disposable 6 can incorporate an adhesive backing to attach the system to the patient. Seals can incorporated across the input interface 50 and output 70 for assuring sterility, and can be applied under vacuum while the reservoir and metering chamber are in their empty configuration to minimize the amount of air left in the system after filling.

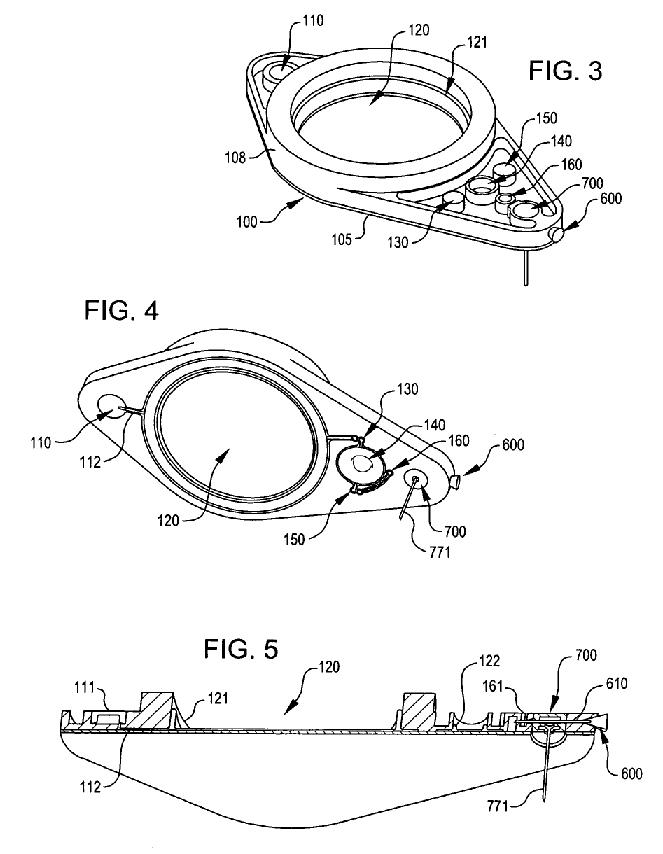

Turning now to FIG. 3, a top perspective view of an exemplary disposable device 100 is shown. The metering system of FIG. 3 operates using the following assemblies which are mated during use: a disposable device, a sequencer/monitor, and a cannula assembly. In one embodiment, the disposable device can connect with the cannula assembly through various mechanisms such as through an output needle, among others.

The device 100 has an input interface 110 that communicates with a reservoir 120 for storing medication. The reservoir 120 encompasses the variable volume created between the rolling bellows 121 and the disposable base plate 105 and in some embodiments the disposable frame 108, and fills a metering chamber 140 through an input valve 130. The metering chamber 140 in turn is in fluid communication with an output valve 150. The output valve 150 allows fluid to flow to an output chamber 160. The output chamber 160 is adapted to engage a cannula assembly 700. When the user connects or interfaces the disposable device 100 to the cannula assembly 700 by inserting an output interface needle 600 into an opening 601 on the device 100 (as seen in cross section in FIG. 5), the output chamber 160 is in fluid communication with the cannula assembly 700 through the needle portion of the output interface needle 600.

The cannula assembly 700 dispenses medication through an elongated arm or a skin penetrating member 771 such as a needle, micro-needle or cannula, among others. Additionally, the device 100 can receive a second medication through a septum 710 (FIG. 6) at the top of the cannula assembly 700. In this manner, the patient can use the cannula assembly 700 to dispense a second medication beneath his or her skin 8 using the same skin penetrating member 771.

Reference may be collectively had to FIGS. 3-7 for the following discussion. FIG. 4 shows a bottom view of the device 100 with the base plate 105 removed. The device 100 has a fluid path 112 that allows medication from the input interface 110 to fill the reservoir 120. The device 100 also has a fluid path that allows a metering chamber 140 to fill through the input valve 130. The metering chamber 140 in turn is in fluid communication with the output valve 150, which in turn allows fluid to flow to the output chamber 160. The output chamber 160 is in fluid communication with the cannula assembly 700 via output interface needle 600 to provide medication through the skin penetrating member 771.

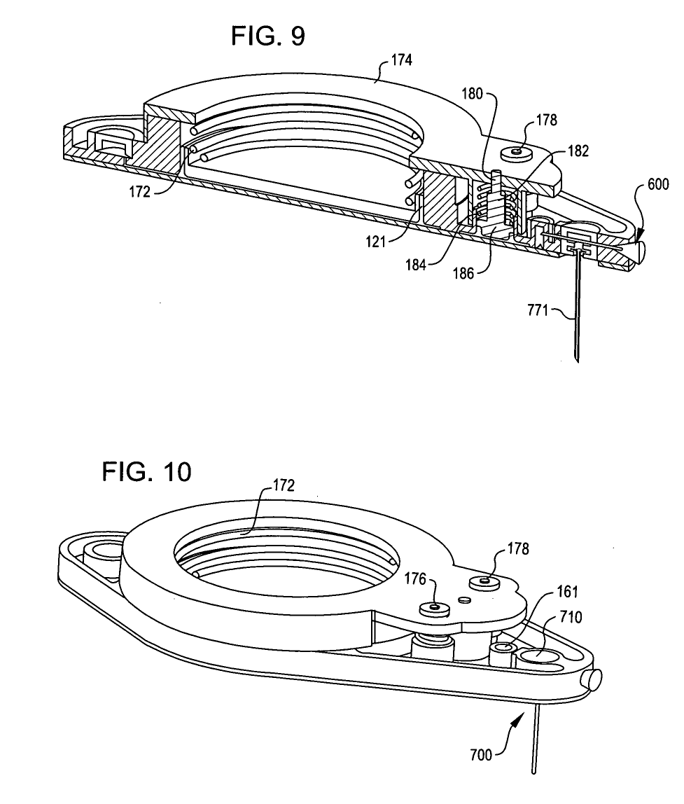

FIGS. 5 and 6 show bottom and top perspective cross-sectional views of the device 100. As shown therein, an input interface septum 111 accepts an external refill input such as a syringe needle that can be used to recharge the reservoir 120. In one embodiment, the patient uses a syringe to inject medication through the interface septum 111. The user can completely fill the reservoir 120, or can fill a portion thereof. A piston plate (not shown) and a rolling bellows (or rolling diaphragm) 121 partially forming the reservoir 120 are urged upwardly as medication flows into the reservoir 120. The rolling bellows provides a medication chamber with flexible sides for containing medication and a pressure source to drive the expulsion of medication upon patient or user command. In one embodiment, the rolling bellows is thin-walled and moves in an annular space. The rolling bellows 121 is secured on a portion of the body of the reservoir 120 and is arranged in a sealing and axially displaceable manner. The rolling bellows is downwardly urged by a spring interacting with a piston plate 173 (FIG. 9).

Similarly, the metering chamber 140 has a rolling bellows or rolling diaphragm 141. In alternative embodiments, the rolling bellows or diaphragm 121 or 141 can be replaced by a piston and O-ring seal combination.

To activate the device 100, the patient inserts the output interface or handle 600 into an opening on the device 100. The handle 600 has a needle 610 that is inserted through the cannula assembly 700 and a septum 161. As shown in FIG. 5, the needle 610 has a sharp end that enters the output chamber 160 and a side hole that provides fluid communication between the output chamber 160 and the cannula assembly 700. Once inserted, the handle 600 can be removed by the patient to disengage the device 100 from the cannula assembly 700. The removal of the handle 600 permits the removal of the device 100 while leaving the cannula assembly 700 intact in the patient. In this manner, the device 100 can be connected to the cannula assembly 700 and can be removed multiple times as desired by the patient.

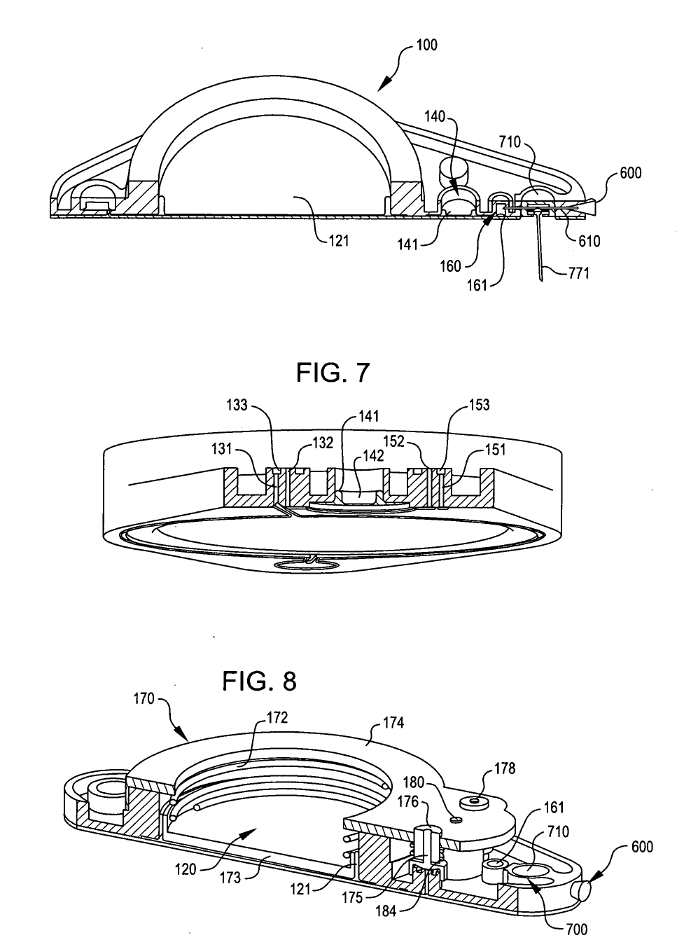

FIG. 7 shows another cross-sectional view of the device 100, with disposable base plate 105 again removed, across the midline of the valves and metering chamber. The output valve 150 is formed of a center port 151 which is in fluid communication with an outer port 152 when valve diaphragm 153 is allowed to move away from the center port 151. Further an input valve 130 is formed of a center port 131 which communicates with an outer port 132 when a valve diaphragm 133 is not pressed against center port 131. When pressure is applied to the diaphragm forcing it against the valve surface, the valve is closed. When the pressure is removed, fluid is allowed to freely flow between the center and outer port 131 and 132, respectively.

In one embodiment, the device 100 operates by scavenging energy provided by the patient or user as medication is dispensed by mechanical activation by the user. This negates the need for electronic actuation to dispense medication. As a result, this embodiment is reliable and cost effective without the complications and expense of electronics and associated batteries and rechargers.

The input interface 110 receives medication from a syringe in one embodiment. The input interface 110 delivers medication through a channel shown in FIG. 4 to the reservoir 120. The reservoir is pressurized by a plate that is spring loaded in the sequencer/monitor 4. The medication from the reservoir 120 in turn flows through a channel to the input valve 130.

In the resting state all valves may be closed affording additional safety to the user. Upon user actuation (FIG. 4), valves 130 and 150 are sequenced as follows. The input valve 130 is opened while the output valve 150 is maintained closed, allowing medication to flow into the metering chamber 140. The input valve 130 is then closed upon the completion of the filling of metering chamber 140. At this point the output valve 150 is opened, thereby allowing medication to flow from the metering chamber 140 through a channel to the output chamber 160. The output valve may then be closed again to restore a resting configuration.

The output chamber 160 is connected (FIG. 6) via the output interface 600 through the cannula assembly septum 710 to the cannula assembly reservoir. The cannula assembly 700 incorporates a cannula 771 that dispenses medication subcutaneously into the patient. In one embodiment, the cannula 771 is inserted using an applicator that injects and withdraws an insertion needle into the patient's skin to install the cannula 771. Once the insertion needle and the cannula 771 are positioned beneath the skin, the insertion needle is removed leaving the cannula 710 in a deployed position and ready to deliver insulin to the patient. More information on a suitable applicator is disclosed in co-pending patent application entitled “Infusion Assembly” filed on May 11, 2007 and having Ser. No. 11/803,007, the content of which is incorporated herein by reference.

The system may include an optional infusion septum or port 710 for delivery of other drugs such as either long acting or short acting insulin. Medication delivered through the optional infusion septum or port 710 is isolated from medication in the rest of the system by the output valve 150. The optional port 710 of the cannula assembly 700 allows a second liquid medicament, such as fast acting insulin, to be delivered at meals, for example. The fast acting insulin may be directly injected into the septum 710 on top of the cannula assembly 700 using a syringe and the fast acting insulin then enters the septum for delivery through the cannula assembly reservoir 730.

The system can provide control over how much of an insulin dosage is to be delivered by having the patient depress the button or actuator a desired number of times. For example, if the metering chamber has a capacity of 0.5 units, each actuation can deliver 0.5 units of insulin and if three units of insulin are desired, six actuations will deliver the desired amount.

The reservoir 120 receives medication through the input septum 111 and the received medication is stored in the reservoir 120. To fill the reservoir 120, in one embodiment, the user moves a syringe plunger a few times to ensure that bubbles are removed from the reservoir. In another embodiment, the reservoir 120 is made of a clear material or otherwise visible so the patient can inspect the amount of medication stored by the reservoir as well as any bubbles therein. The visible reservoir 120 is advantageous in that the patient can determine if he or she has a sufficient amount of medication to last the patient through a particular trip.

Upon user actuation, medication to be dispensed is allowed to move through the input valve 130 into the metering reservoir 140 which has a precisely controlled maximum volume. After the metering reservoir is filled to its maximum volume the medication in the metering reservoir 140 is then allowed to flow through an output valve and delivered through an output septum. The output septum provides the medication through an output needle across a septum in a cannula assembly. The medication is then delivered to the patient through a cannula.

FIGS. 8-10 show one exemplary sequencer/monitor device 170 that operates as a system with the disposable device 100 of FIG. 3. As shown in FIG. 8, a substantially cylindrical spring 172 engages a piston plate 173 and exerts force on the medication contained inside the bellows 121. The spring 172 is bounded by a top plate 174. Another energy storage device (such as a coil spring 440 (FIG. 29)) can be used to move valves in a predetermined sequence. Each energy storage device can operate stand-alone, or alternatively, the two energy storage devices can operate in tandem. The energy required to deliver the fluid or medication is stored as mechanical energy and transferred to the reservoir, while the energy necessary to sequence the action of delivery can be provided by the same energy source or a secondary source which can be either mechanical or electrical.

In one embodiment, the top plate 174 supports two electrically activated solenoids 176 and 178 that control the input valve 130 and the output valve 150 of FIG. 3, respectively. A piston 186 for the metering chamber 140 has a first portion 180 and a second portion 182, both of which are surrounded by a spring 184. The first portion 180 helps to maintain alignment of the piston as it travels through a hole in top plate 174. The second portion 182, which is larger in diameter then the first portion 180, is designed to run into plate 174 when piston 186 is at its maximum travel corresponding to when the metering chamber 140 is full. Thus the heights of the second arm 182 can be varied to adjust the volume of medication that flows into the metering chamber 140. In another embodiment, a screw can be used to adjust the height differential between portion 182 to vary the volume of medication flowing into the metering chamber 140.

The shaft or core 175 of each of solenoids 176 and 178 can be a permanent magnet. In another embodiment, a suitable material such as barium titanate can be incorporated in the diaphragm membrane in the valve and an electromagnet coil is then used to lift the membrane directly. This embodiment provides a low manufacturing cost and a low profile.

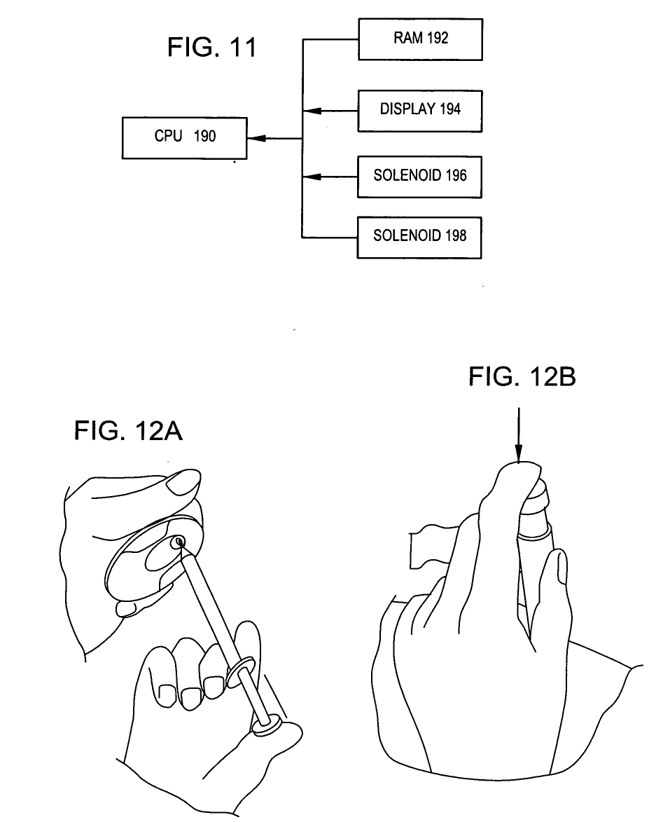

FIG. 11 shows an exemplary circuit to detect dosage administration over a period of time. A microcontroller or CPU 190 receives an input from a button. The CPU 190 is connected to a memory 192 and drives a display 194 to show the dosage and the time period, for example. The CPU 190 is powered by a battery which is not replaceable. Hence, once power runs out, the sequencer is replaced. This configuration is advantageous in that it protects the electronics from water damage and also eliminates the hazards that could arise if the user inserts the battery incorrectly. Alternatively, in another embodiment, the battery can be user-replaceable to minimize replacement cost. The CPU 190 has at least two I/O ports to control solenoids 196 and 198, which correspond to solenoids 176 and 178 of FIG. 8.

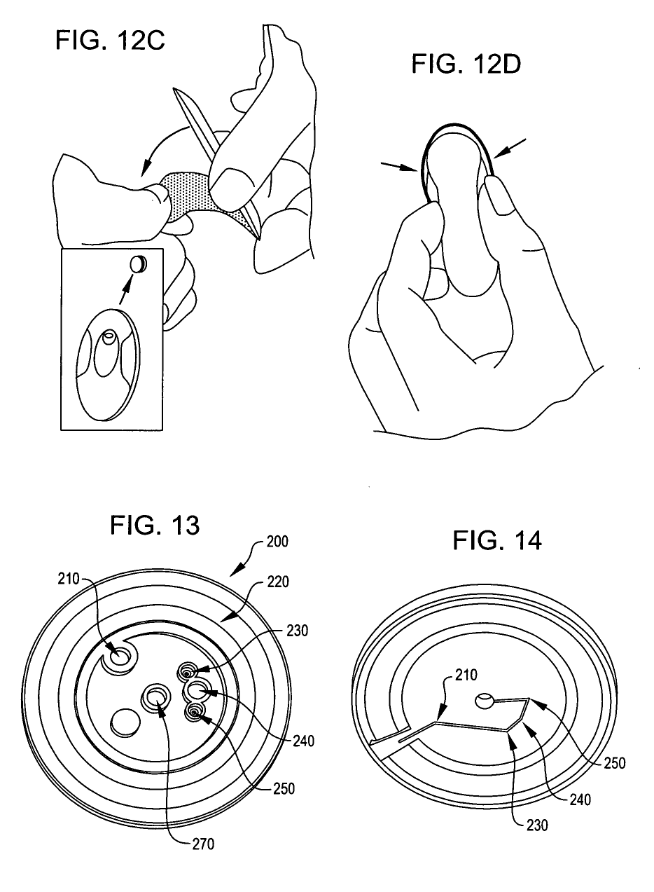

FIGS. 12A-12D show an exemplary process for deploying the device of FIG. 3. Initially, the user locates a filling port, in this case an input interface on the device or dispenser. The dispenser is filled with a desired amount of insulin using a syringe or other suitable filling mechanisms. In one configuration, the patient moves the syringe plunger in and out to purge bubbles in a reservoir in the dispenser. The user confirms the absence of bubbles before proceeding. Next, the user selects an area of clean, dry skin and uses an applicator (shown in FIG. 12B) to deploy the cannula assembly. The patient presses an applicator button to deploy the cannula assembly. After installation, the applicator is then removed. More details on a suitable applicator are disclosed in commonly assigned, co-pending patent application Ser. No. 11/803,007, the content of which is incorporated by reference. Next, in FIG. 12C the user removes a liner to expose the adhesive on the bottom of the dispenser, and mounts the dispenser over the deployed cannula assembly and on the patient's skin in FIG. 12D. A method to count dosage administration over a period of time may include one of shorting two wires together when a button is actuated by a user to manually dispense medication, waking up a processor when the two wires are shorted together, and incrementing a dosage count. Other alternatives for detecting when the button is actuated by the user can be used as well. For example, a magnetically coupled relay can detect button closure, or a switch directly activated by the user to indicate medication dispensing can be used, among others. The system can display the dosage count and the period of time between doses. The system can wake up the processor when a display button is depressed to turn on a backlight to a display. The system can wake up the processor when the display button is depressed and turn on the display. The system can also wake up the processor when the display button is depressed and turn on a backlight to a display when the display button is depressed once, and turn on the backlight when the display button is depressed twice in succession.

In one embodiment, the system can be used to deliver one or more boluses of insulin to the patient over a period of time accompanied prior to ingestion of glucose in the form of a meal. The number of pulses, the amount of insulin in each pulse, the interval between pulses and the amount of time to deliver each pulse to the patient are selected so that total body tissue processing of glucose is restored in the patient.

Turning now to FIG. 13, a top perspective view of a base plate for a second exemplary disposable portion of a medication delivery device 200 is shown. The metering system of FIG. 13 operates using three assemblies which are mated during use: a disposable device, a sequencer/monitor, and a cannula assembly. The device 200 has an input interface 210 that communicates with a reservoir 220. In one embodiment, the reservoir 220 is configured as a compressible ring allowing the control mechanisms to be interfaced within the center of the device. This position for the reservoir 220 enables the largest possible volume of medication to be stored for a given radial length of device.

The reservoir 220 fills a metering chamber 240 through an input valve 230. The metering chamber 240 in turn is in fluid communication with an output valve 250. The output valve 250 allows fluid to flow to a cannula assembly receiver 270. The user or patient connects the disposable device 200 to the cannula assembly 700 (FIG. 19) by interfacing the cannula assembly receiver 270 with the cannula assembly 700. The device 200 can receive a second medication through a septum 710 at the top of the cannula assembly 700. In this manner, the patient can use the cannula assembly to dispense a second medication into his or her skin 8 using the same cannula.

FIG. 14 shows a bottom view of the device 200 with the disposable base plate 205 removed to expose the fluid path that provides medication from the input interface 210 to fill the reservoir 220, and the fluid path that fills a metering chamber 240 through the input valve 230. The metering chamber 240 in turn is in fluid communication with the output valve 250, which in turn allows fluid to flow to the cannula assembly receiver 270. The cannula assembly receiver 270 is in fluid communication with the cannula assembly 700 to provide medication through a cannula 771, for example.

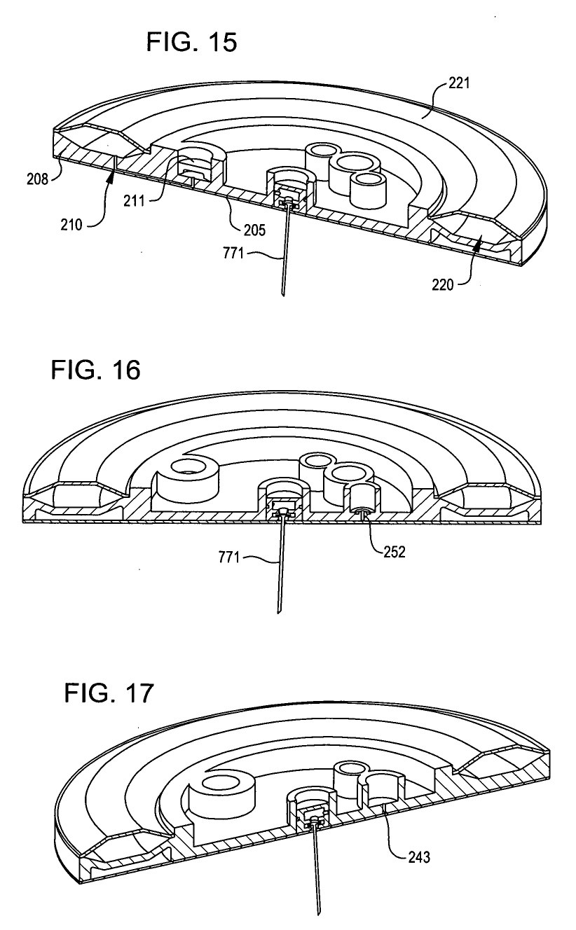

FIGS. 15 and 16 show two cross-sectional views of the device 200 of FIG. 13. The input interface 210, used to recharge the reservoir 220, incorporates a septum 211 that accepts an external refill input from a syringe and needle. Diaphragm or wall 221 forming half of the reservoir 220 is urged upwardly as medication flows into the reservoir 220. The wall or diaphragm 221 is semi-rigid and may store activation energy resulting from the filling of the reservoir. The diaphragm 221 is secured on a portion of the body of disposable frame 208 creating the reservoir 220.

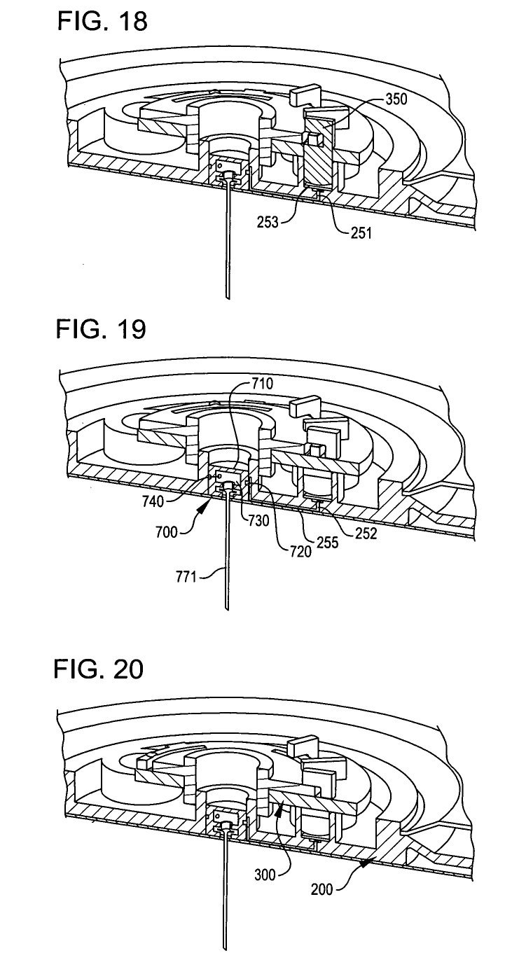

FIGS. 17-20 show cross-sectional views illustrating the operation of the output valve 250 of the device 200. In FIG. 17, medication in the output valve 250 flows into the cannula assembly 700 through a metering chamber port 243. FIG. 18 shows plunger 350 in the valve open position allowing medication flow through the output valve to the cannula assembly 700. In the closed position the output valve plunger 350 is urged against a valve diaphragm 253. When the output valve plunger 350 is lifted, medication is allowed to flow beneath the valve diaphragm 253 from a center port 251 to an outer output port 252.

As best shown in FIG. 19, fluid flows through an output port 252 through a channel 255 into a cannula assembly input channel 720 of the cannula assembly 700. A cannula assembly septum 710 maintains medication inside the cannula assembly 700. Medication then flows into a cannula assembly input port 740 and then is delivered to the patient through a cannula, a micro-needle or a suitable dispensing device 771. In FIG. 20 the plunger 350 is shown in the closed position.

The device may be configured such that it is manually actuated by the patient or user, namely that medication dispensing is powered by mechanical activation by the user and no electronic actuation is used to dispense medication.

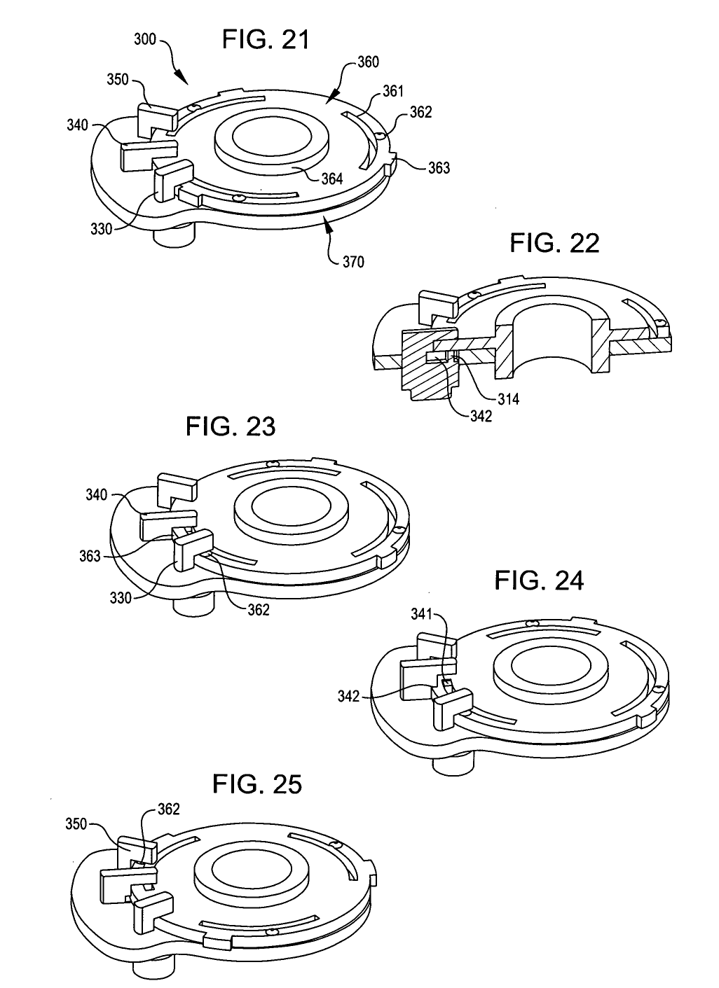

FIGS. 21-25 show an exemplary valve control system 300 for the disposable dispensing device 200. Turning now to FIG. 21, the valve control system 300 has a valve control plate 360 and a valve control guide plate 370. The valve control guide plate 370 is fixed, while the valve control plate 360 moves with respect to control elements, 330, 340, 350, and valve control guide plate 370. The valve control plate 360 has a plurality of fill relief openings 361, valve actuation buttons 362, and sequence stoppers 363.

Projecting through the valve control guide plate 370 are an input valve plunger 330, a metering chamber plunger 340, and an output valve plunger 350. Plungers 330-350 move in sequence to control and meter the flow of medication to the patient. The sequencing of the plungers 330-350 is achieved through the sequenced interaction of the fill relief openings 361, valve actuation buttons 362, and sequence stoppers 363. Adjacent each stopper 363 is a valve actuation button 362, and a fill relief opening 361. The button 362 can be conical shaped, pyramidal shaped or any other suitable shape that lifts each of the valve plungers 330 or 350 up and allows them to return to their rest or down position. FIG. 21 shows the valve control plate 360 in a rest position. In this position both valve plungers 330 and 350 are in their closed positions, thereby precluding any flow through the system.

FIG. 22 shows in more detail the relationship between the fill relief 361 and the metering plunger 340. In the example of FIG. 22, the plunger 340 has an empty plunger stopper 341 and a full plunger releaser 342 that engage the fill relief 361 and sequence stopper 363, respectively. The operation of the stopper 341 and the release 342 is illustrated next in FIGS. 23-25.

The operation of the system of FIGS. 23-25 with respect to the plungers 330, 340 and 350 will be discussed next. Referring now to FIGS. 21-25, the valve control plate 360 rides on the valve control guide plate 370. As the plate 360 rotates, the sequence stopper 363 bumps the edge of the meter chamber plunger 340. As the valve actuation button 362 moves, it lifts the input valve plunger 330, opening the input valve (FIG. 23) so that fluid can enter the metering chamber. The meter chamber plunger empty stopper 341 moves up into the fill relief slot 361. As shown in FIG. 24, the metering chamber plunger 340 completes its ascension and the meter chamber plunger full releaser 342 is aligned with the stopper 363 and allows the control plate 360 to continue to rotate. As shown in FIG. 25, the bump 362 passes the input valve plunger 330 allowing the input valve to close. Valve plate 360 continues to rotate until the empty stopper 341 runs into the trailing edge of the fill relief opening 361. at this point the valve actuation button 362 moves under the output valve plunger 350 and opens the output valve to allow fluid to escape from the metering chamber. Next, the metering chamber plunger 340 drops down until the empty stopper 341 no longer runs on the trailing edge of the slot 361. This allows the valve control plate to rotate until the valve actuation button 362 moves out from under the output valve plunger 350, allowing the output valve to close. In this embodiment, the device receives sufficient charge for one cycle and comes to a rest position until the next user dispensing request causes a repeat of the actuation of each valve in the above predetermined sequence to dispense the fluid into the user through his or her skin.

From a valve perspective, the operation of each valve in a predetermined sequence to dispense the fluid will be discussed next. Upon user actuation, valves 230 and 250 are sequenced as follows. The input valve 230 is opened by lifting the plunger 330 while the plunger 350 of the output valve 250 is maintained in its rest or closed position. The input valve 230 is then closed, allowed to return to its rest position, upon the completion of the filling of metering chamber 240. At this point the input valve 230 is closed and the output valve 250 is opened by lifting the plunger 350, thereby allowing medication to flow from the metering chamber 240 through a channel to the output chamber 270. The output chamber 270 is interfaced with the cannula assembly septum 710. The infusion chamber 730 incorporates a cannula 771 that dispenses medication subcutaneously into the patient. Once an insertion needle (not shown) and the cannula 771 are positioned beneath the skin, the insertion needle is removed leaving the cannula 710 in a deployed position and ready to deliver insulin to the patient.

As discussed above, the system includes an optional infusion septum or port 710 similar to the septum 111 as part of an insertion set for delivery of other drugs such as either long acting or short acting insulin. The system can also provide control over how much of each particular insulin dosage is determined to be delivered by having the patient depress the button or actuator a desired number of times. For example, if each actuation can deliver 0.5 units of insulin and if three units of insulin are desired, six actuations can deliver the desired amount.

The reservoir 220 receives medication from the input septum and the received medication is stored in the reservoir 220. To fill the reservoir 220, in one embodiment, the user moves a syringe plunger a few times to ensure that bubbles are removed from the reservoir. The reservoir 220 may be made of a clear material so the patient can inspect the amount of medication stored in the reservoir as well as entrapment of bubbles therein. The visible reservoir 220 is advantageous in that the patient can determine if he or she has a sufficient amount of medication to last the patient through a particular trip.

Upon user actuation, medication to be dispensed is allowed to move through the input valve 230 into the metering reservoir 242 which has a precisely controlled volume. The medication in the metering reservoir 242 is then allowed to flow through an output valve and delivered through the cannula assembly. The medication is then delivered to the patient through a cannula. The reservoir and the insertion set may provide the fluid control components while the sequencer/monitor may provide the motive power and monitoring capabilities. The motive power is stored as mechanical energy in the reservoir, while the energy necessary to sequence the action of delivery can be provided by the storage reservoir or a secondary source such as an additional spring.

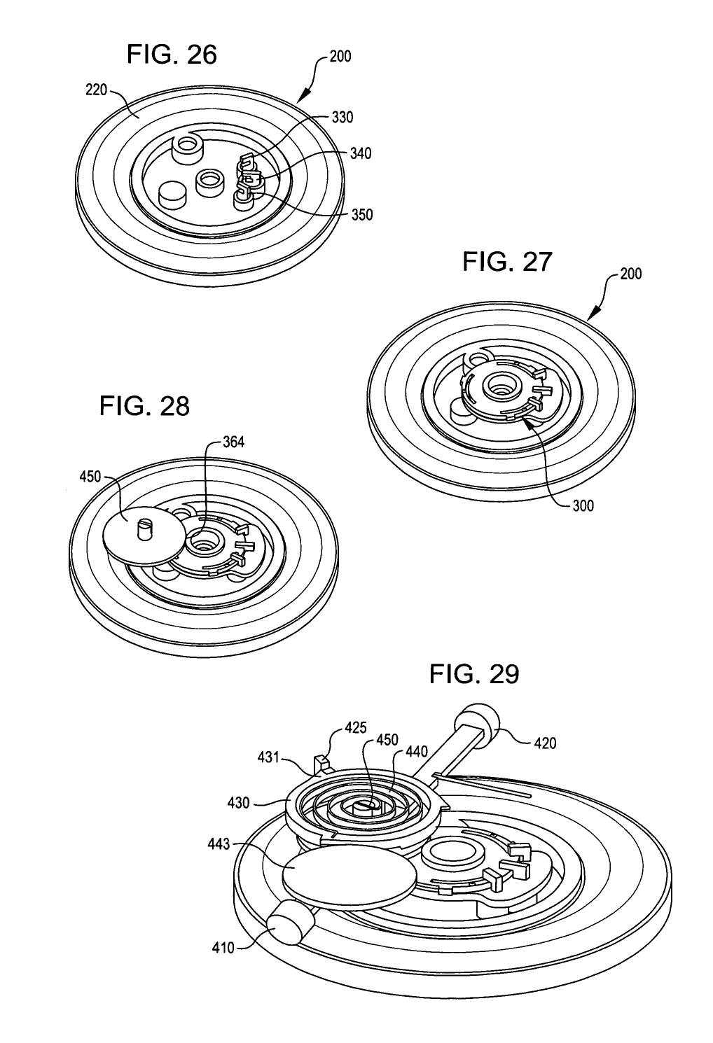

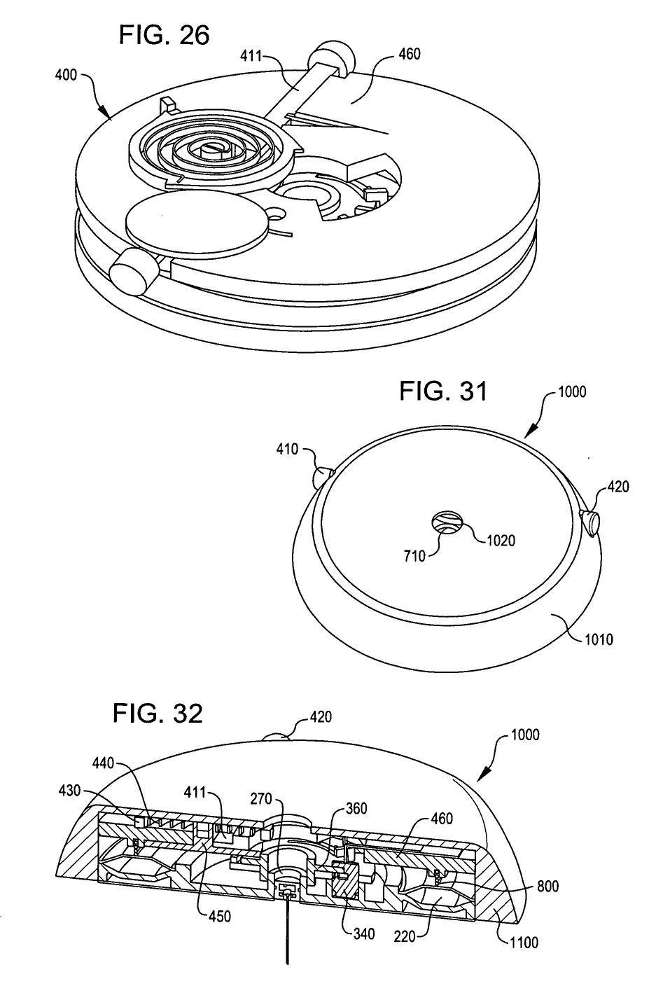

FIGS. 26-35 illustrate the assembly of one embodiment incorporating mechanisms for receiving, storing and dispensing mechanical energy to dispense medication to the patient. The power to lift and sequence the plungers 330, 340 and 350 is captured from the patient's physical motion in a manner that is similar to winding up a mechanical watch. Energy is stored by winding a spring, and upon user command, energy is released to operate the plungers 330, 340 and 350 to move medication from the reservoir 220 to the cannula assembly 700 for delivery.

FIG. 26 is a top view of the base plate of the device 200 showing the reservoir, the metering chamber, the input valve, the output valve, and the cannula assembly receiver, among others. FIG. 27 shows the valve control system 300 mounted on the base plate of FIG. 26.

FIG. 28 shows the first element in an energy storage system 400. The system 400 includes an energy transfer device 450 in contact with the drive surface 364.

The user interface control 400, and valve control system 300 are added to the disposable device 200 and shown in varying detail in FIGS. 29-35. As shown in FIG. 29, an energy storage device 440, such as a spring, is used to store the energy, provided by the user or patient, and required to carry out the actions of the user interface control. The energy transfer device is charged through an energy delivery button or an energy input member 410, which acts as a winder. The winder energy input member 410 travels along a guide path 411 and when pushed, spins energy transfer device 443, which in turn spins drive ring 430. The linear travel of energy input member 410 is such that when pushed to its stopping point, the radial displacement transferred to the drive ring 430 is greater then 120 degrees. In this way, drive ring 430 is rotated enough to allow a drive ring catch 431 to engage with the drive ring stop 425. This action in turn stores energy in the energy storage device 440. The spring 440 can then release its energy into energy transfer device 450 which interfaces with and drives the valve control system 300 as described previously. This provides one half of the interlocked user interface control. A drive ring release button 420, which locks drive ring 430 until pushed, forms the other half of the interlocked user interface control.

In FIG. 30, user interface control components are shown as mounted on the user interface control mounting plate 460. As shown in FIG. 31, the device 1000 is enclosed in a medication dispensing housing 1010 which includes openings to accommodate the energy input member 410 and the drive ring release button 420 along with cannula assembly septum 710 access hole 1020. FIG. 32 shows a cross-sectional view of the entire device 1000 in the housing 1010. In FIG. 32, a cross sectional view of the device 1000, tapered walls 1100 provide structural support as well as a large base to secure the device 200 to the patient while maintaining a slim profile. The system of FIG. 32 provides a low profile insulin pump manually activated by the user which is small enough to hide under the user's clothing. The system also provides a convenient, secure and inconspicuous user interface to dispense medication as needed.

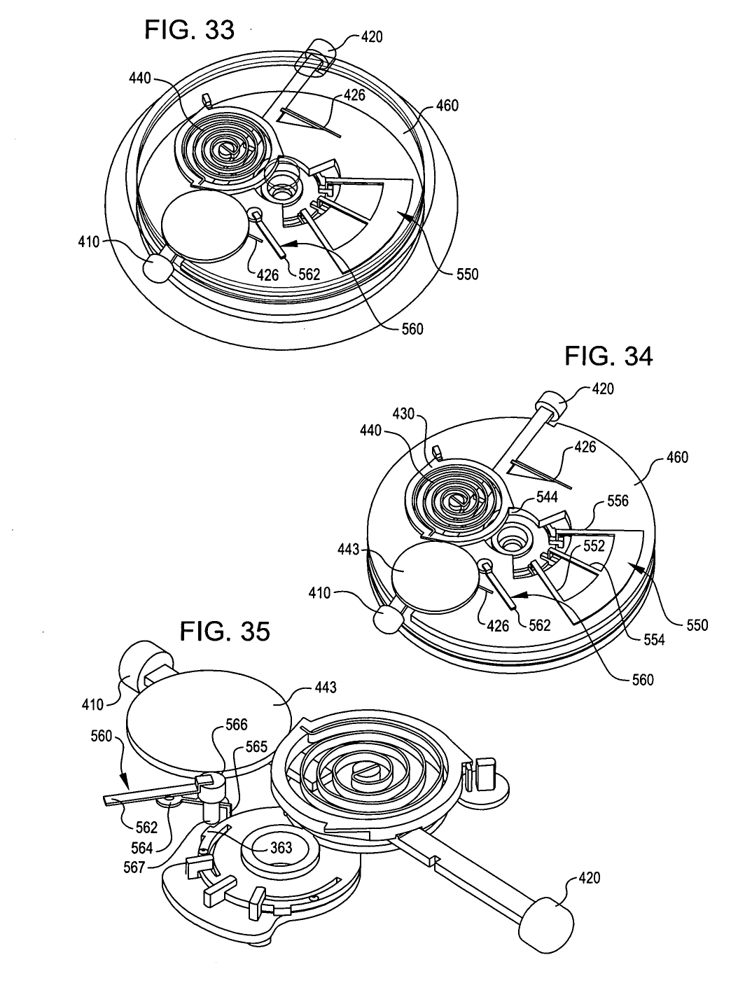

FIGS. 33-35 illustrate additional features of the device 1000. In FIG. 30, additional spring components associated with the operation of device 1000 are shown as mounted on the user interface control mounting plate 460 beneath the housing 1010. These include button return springs 426 which interface with the buttons 410 and 420 and hold them in their rest positions. Valve and meter plunger control spring 550, which maintains the associated elements in their rest positions. A user feed back unit 560 incorporating spring 562 is also shown.

In FIG. 34 the housing 1010 has been removed and elements of spring 550 have been further defined.

In FIG. 35, the feedback unit 560 includes a feedback unit hammer 566 mounted under a feedback unit spring 562. The hammer 566 generates audible or tactile feedback information to the user or patient at the end of a delivery cycle. The feedback unit 560 includes a feedback unit release 564 that engages threaded shaft 567 of feedback unit hammer 566. At the beginning of a delivery cycle, as energy transfer device 443 is rotated, as described above, it also rotates feedback unit hammer 566. This action causes the threaded shaft 567 of the feedback unit hammer 566 to ride up on the feedback unit release device 564, and load feedback unit spring 562. At the end of the delivery cycle, sequence stopper 363 rotates into feedback hammer release 564, release tab 565. This in turn forces the feedback hammer release 564 away form the threaded shaft 567, releasing hammer 566. Hammer 566, once released, is driven into the user interface control mounting plate 460 by spring 562. This action creates both an audible sound and a tactile vibration.

The invention has been described in terms of exemplary embodiments, it is contemplated, however that the invention include variations within the scope of the appended claims. For example, it is contemplated that the invention may be realized with mechanical or with a combination of electrical and mechanical sub-systems. Many of the parts, components, materials and configurations may be modified or varied, which are not specifically described herein, may be used to effectively work the concept and working principles of this invention. They are not to be considered as departures from the invention and shall be considered as falling within the letter and scope of the following claims.

What is claimed is:

1. An apparatus to store and dispense a fluid on-demand, comprising:
a reservoir;
an energy storage device coupled to the reservoir to store energy, wherein the energy is mechanically generated by a user's action;
a cannula assembly adapted to be positioned on a patient and coupled to the reservoir prior to use; and
a sequencer coupled to the reservoir and the energy storage device to allow the fluid to be dispensed upon command.

2. The apparatus of claim 1, comprising a metering assembly coupled to the reservoir.

3. The apparatus of claim 2, wherein the metering assembly comprises:
a. an input valve;
b. a metering chamber coupled to the input valve; and
c. an output valve coupled to the metering chamber.

4. The apparatus of claim 1, wherein the reservoir is configured as a ring.

5. The apparatus of claim 4, wherein the reservoir comprises a rolling bellows.

6. The apparatus of claim 1, comprising a metering assembly coupled to the reservoir, wherein the metering assembly is encircled by the reservoir.

7. The apparatus of claim 1, wherein the energy storage device stores energy when the reservoir is filled or when the reservoir is placed on the patient.

8. The apparatus of claim 1, wherein the energy storage device comprises a spring wound or compressed by the user's action.

9. The apparatus of claim 1, comprising an interlocked user interface adapted to be actuated by a user.

10. The apparatus of claim 9, wherein the interlocked user interface comprises an energy delivery button and an energy release button.

11. The apparatus of claim 10, wherein the energy delivery button charges an energy storage device.

12. The apparatus of claim 10, wherein the energy delivery button winds or compresses a spring.

13. The apparatus of claim 10, wherein the release button allows energy to be discharged from a dispensing energy storage device to dispense the fluid.

14. The apparatus of claim 9, comprising:
a. an energy delivery button to wind a spring;
b. a release button to allow energy to be released from the spring; and
c. a metering assembly coupled to the reservoir, wherein the metering assembly comprises:
i. an input valve;
ii. a metering chamber coupled to the input valve; and
iii. an output valve coupled to the metering chamber,
wherein the spring provides energy to actuate each valve in a predetermined sequence to dispense the fluid.

15. The apparatus of claim 14, wherein the spring provides energy to activate the metering chamber.

16. The apparatus of claim 14, comprising a metering spring to provide energy to activate the metering chamber.

17. The apparatus of claim 1, comprising a dispensing feedback unit to indicate fluid dispensing.

18. The apparatus of claim 1, wherein the cannula assembly comprises one of: a cannula, a micro-needle, a needle.

19. The apparatus of claim 1, comprising a receiving port to receive a second medication.

20. An apparatus to store and dispense a fluid on-demand, comprising:
a reservoir;
a metering assembly coupled to and powered by the reservoir;
an energy storage device coupled to the reservoir to store energy, wherein the energy is mechanically generated by a user's action;
a cannula assembly adapted to be positioned on a patient and coupled to the reservoir prior to use; and
a sequencer coupled to the reservoir, the metering assembly, and the energy storage device to control fluid dispensing upon command.
